(19) United States
(12) Patent Application Publication
(10) Pub. No.: US 2024/0300881 A1
(43) Pub. Date: Sep. 12, 2024

(54) PROCESS FOR PREPARATION OF LITHIUM L-THREONATE AND USE THEREOF

(71) Applicant: XIXIOXOME BIOTHERAPEUTICS, New Hope, PA (US)

(72) Inventor: Philip SJOSTEDT, Doylestown, PA (US)

(21) Appl. No.: 18/597,745

(22) Filed: Mar. 6, 2024

Related U.S. Application Data
(60) Provisional application No. 63/489,446, filed on Mar. 10, 2023.
(60) Provisional application No. 63/465,775, filed on May 11, 2023.

(51) Int. Cl. C07C 51/295; A61K 31/191

(52) U.S. Cl. CPC C07C 51/295; A61K 31/191

(57) ABSTRACT

A composition of lithium L-threonate and a pharmaceutical composition of an effective amount of lithium L-threonate and pharmaceutically acceptable excipients. A method of making lithium L-threonate, by reacting calcium L-threonate with Li2SO4 and Ba(OH)2, and producing lithium L-threonate. A method of making lithium L-threonate, by reacting calcium L-threonate with Li2SO4, and producing lithium L-threonate. A method making lithium L-threonate, by synthesizing L-threonic acid, reacting the L-threonic acid with Li2SO4, and producing lithium L-threonate. A method of treating bipolar disorder, by administering an effective amount of lithium L-threonate to a patient, and treating bipolar disorder. A method of treating a disease or disorder, by administering an effective amount of lithium L-threonate to a patient, and treating the disease.

### BACKGROUND

### 1. Technical Field

The present invention relates to compositions and methods for synthesizing lithium L-threonate and its use in health care.

### 2. Background Art

Bipolar disorder ranks high among the most prevalent and frequently underdiagnosed and undertreated affective disorders in the United States. Characterized by recurrent episodes of mania, depression, and mixed states, the annual economic burden of bipolar disorder—including healthcare costs and lost productivity of patients and their caregivers—was estimated at $24 billion in the United States in 1998.

Despite the growth in research in recent years, an estimated 69% of people with bipolar disorder have been misdiagnosed; most notably, patients with bipolar disorder had received a mean of 3.5 other diagnoses and have consulted 4 physicians before being accurately diagnosed. Bipolar disorder increases the risk for suicide and is associated with several psychiatric and medical comorbidities, including substance misuse, anxiety disorders, cardiovascular disease, metabolic syndrome, and hypertension.

An increasing body of evidence suggests that when appropriate pharmacotherapeutic interventions are initiated early in the course of the disorder, the ultimate prognosis is substantially improved. Longitudinal data on the management of patients with bipolar disorder, including monitoring for efficacy, safety, adherence, and comorbidities, are available to provide practical, clinically applicable information.

The pathophysiology of mood disorders is complex, and a focus on the correction of neurochemical deficits alone has proven simplistic. Conventional medications used for bipolar disorder mediate their effects through complex biochemical cascades. The various mood stabilizers available to clinicians-including lithium and anticonvulsants such as carbamazepine, valproic acid, and lamotrigine-operate through neural signaling pathways, the clinical implications of which are as yet unknown. Translational research efforts are underway to delineate precisely how mood stabilizing agents influence neurotransmitter receptors and their respective intracellular signaling pathways that affect gene expression, cell differentiation, proliferation and death, and the structure and function of nerve cell activity. Lithium is an effective mood stabilizer that is used principally for the management of bipolar disorder, and is approved in various countries as lithium gluconate, lithium acetate, lithium glutamate, and lithium carbonate.

There remains a need for a treatment for bipolar disorder that is more effective, a definite and controllable structure, easily absorbed in the human body, supply lithium more effectively for patient needs, and avoid side effects associated with the current formulations of lithium.

### SUMMARY OF THE INVENTION

The present invention provides for a composition of lithium L-threonate.

The present invention provides for a method of making lithium L-threonate, by reacting calcium L-threonate with Li2SO4 and Ba(OH)2, and producing lithium L-threonate.

The present invention provides for a method of making lithium L-threonate, by reacting calcium L-threonate with Li2SO4, and producing lithium L-threonate.

The present invention provides for a method making lithium L-threonate, by synthesizing L-threonic acid, reacting the L-threonic acid with Li2SO4, and producing lithium L-threonate.

The present invention provides for a pharmaceutical composition of an effective amount of lithium L-threonate and pharmaceutically acceptable excipients.

The present invention provides for a method of treating bipolar disorder, by administering an effective amount of lithium L-threonate to a patient, and treating bipolar disorder.

The present invention provides for a method of treating a disease or disorder, by administering an effective amount of lithium L-threonate to a patient, and treating the disease, wherein the disease is chosen from Lewy-body dementia (LBD), Alzheimer's disease (AD), Alzheimer's disease related dementias (AD/ADRD), behavioral and psychological symptoms of dementia (BPSD), agitation in Alzheimer's disease (AAD), mania, or schizoaffective disorder.

### DESCRIPTION OF THE DRAWINGS

Other advantages of the present invention are readily appreciated as the same becomes better understood by reference to the following detailed description when considered in connection with the accompanying drawing wherein:

FIG. 1 is a chemical structure of lithium L-threonate.

### DETAILED DESCRIPTION OF THE INVENTION

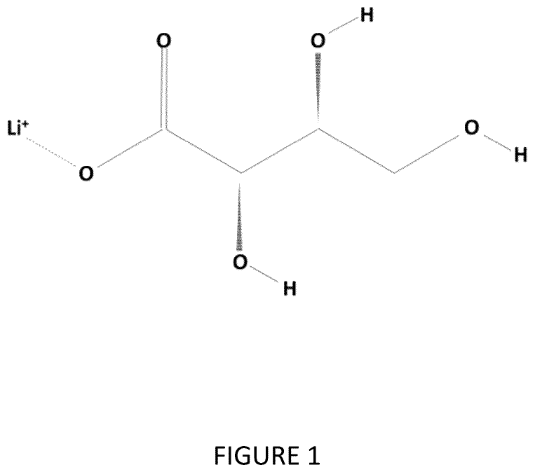

The present invention provides for a new type of lithium (I) of a composition of lithium L-threonate, which has a molecular formula of Li(C4H3O5) and the chemical structure shown in FIG. 1. The use of lithium L-threonate can avoid adverse effects associated with the currently used treatment of lithium carbonate for bipolar disorder.

L-threonic acid is one of main degradation products of ascorbic acid/Vitamin C and has been utilized to develop various chemical formulations, including magnesium L-threonate, calcium-L-threonate, and L-threonate ferrous. In the present invention, L-threonic acid and lithium (I) are combined to produce lithium L-threonate. In each situation, the utilization of L-threonic acid has demonstrated an enhanced absorption of the elemental ion.

There are two synthesis methods of lithium L-threonate.
1. Using L-threonic acid as raw material to produce the lithium L-threonate. Most generally, this method includes synthesizing L-threonic acid, reacting the L-threonic acid with Li2SO4, and producing lithium L-threonate. This method is further described in EXAMPLE 3.
2. Using calcium L-threonate as raw material to produce the lithium L-threonate. It is possible to take ascorbic acid/Vitamin C as the raw material to synthesize calcium L-threonate, then derive lithium L-threonate from calcium L-threonate through replacement of mild acid or cation exchange resin. It is also possible to take calcium L-threonate as raw material and derive the lithium L-threonate directly. As detailed in EXAMPLE 1, the present invention provides for a method of making lithium L-threonate, by reacting calcium L-threonate with Li2SO4 and Ba(OH)2, and producing lithium L-threonate Further, as detailed in EXAMPLE 2, the present invention provides for a method of making lithium L-threonate, by reacting calcium L-threonate with Li2SO4, and producing lithium L-threonate.

The composition of lithium L-threonate, can be used as a treatment for patients with bipolar disorder, including Bipolar Disease, Bipolar I Disorder, Bipolar II Disorder, Bipolar Depression, and Bipolar Mania.

The composition can be used by itself as well as in combination with other compounds. For example, the composition can be used in combination with mood stabilizers, atypical antipsychotics, antidepressants, or anxiolytics.

Oral administration can be preferred, but any other administration method as described below can be used. Any suitable dose can be used, such as that which provides 300-2000 mg of lithium per day.

Therefore, the present invention provides for a method of treating bipolar disorder, by administering an effective amount of lithium L-threonate to a patient, and treating bipolar disorder.

The present invention also provides for a method of treating a disease or disorder, by administering an effective amount of lithium L-threonate to a patient, and treating the disease, wherein the disease is chosen from Lewy-body dementia (LBD), Alzheimer's disease (AD), Alzheimer's disease related dementias (AD/ADRD), behavioral and psychological symptoms of dementia (BPSD), agitation in Alzheimer's disease (AAD), mania, or schizoaffective disorder. Treating the disease or disorder can result in reducing and/or eliminating any symptoms associated with the disease or disorder. Any administration methods described herein can be used, and any suitable dose can be used, such as that which provides 300-2000 mg of lithium per day.

Lewy-body dementia (LBD) is a disease wherein abnormal deposits of alpha-synuclein protein (i.e. Lewy bodies) area found in the brain. The Lewy bodies affect neurotransmitters (acetylcholine and dopamine) in the brain causing problems with thinking, movement, behavior, and mood.

Alzheimer's disease (AD) is a brain disorder wherein memory and thinking skills are slowly destroyed due to the presence of amyloid plaques and tau tangles as well as the loss of connections between neurons, and is the most common cause of dementia in order adults. Nunes, et al. (Curr Alzheimer Res. 2013 January; 10(1):104-7) has shown that a microdose of 300 μg of lithium administered once daily on AD patients for 15 months showed no decreased performance in a mini-mental state examination test compared to lower scores observed for the control group during the treatment. Straten, et al. (Curr Alzheimer Res. 2011 December; 8(8):853-9) describes that induction of neurotrophins due to lithium could be the mechanism of action in treating AD. Leyhe, et al. (J Alzheimers Dis. 2009; 16(3):649-56) describes that AD patients have been found to have a decrease in brain-derived neurotrophic factor (BDNF) and in AD patients treated with lithium, a significant increase of BDNF serum levels and a significant decrease of ADAS-Cog sum scores in comparison to placebo-treated patients were found. Forlenza, et al. (Br J Psychiatry. 2011 May; 198(5):351-6) describes that lithium treatment was associated with a significant decrease in CSF concentrations of P-tau in individuals with amnestic mild cognitive impairment.

Alzheimer's disease related dementias (ADRD) result in the impaired ability to remember, think, or make decisions and interferes with an individual being able to do their everyday activities.

Behavioral and psychological symptoms of dementia (BPSD) are the associated neuropsychiatric symptoms of dementia. These can include cognitive/perceptual symptoms (delusions, hallucinations), motor symptoms (pacing, wandering, repetitive movements, physical aggression), verbal symptoms (yelling, calling out, repetitive speech, verbal aggression), emotional symptoms (euphoria, depression, apathy, anxiety, irritability), and vegetative symptoms (disturbances in sleep and appetite).

Agitation in Alzheimer's disease (AAD) is a symptom of AD that is found as the disease gets worse in an individual. The individual can be restless or worried, and agitation can result in pacing, sleeplessness, or aggression. Devanand, et al. (Contemp Clin Trials. 2018 August; 71:33-39) describes that lithium is an established treatment for bipolar and other psychotic disorders in which agitation can occur. Devanand, et al. (Am J Geriatr Psychiatry. 2022 January; 30(1):32-42) describes a trial of providing low-dose lithium carbonate for treating AAD and it was concluded that low-dose lithium carbonate was not efficacious in treating agitation but was associated with global clinical improvement.

Mania is defined as a period of one week or more in which a person experiences behavioral changes that drastically affects their functioning. Symptoms of mania include increased talkativeness, rapid speech, a decreased need for sleep, racing thoughts, distractibility, an increase in goal-directed activity, psychomotor agitation, elevated or expansive mood, mood liability, impulsivity, irritability, or grandiosity. Mania has been treated with risperidone, olanzapine, haloperidol, lithium, quetiapine, aripiprazole, valproic acid, carbamazepine, and ziprasidone. Akbarzadeh, et al. (Int Clin Psychopharmacol. 2022 Mar. 1; 37(2):54-59) investigated the relationship between personality profile and the response to lithium carbonate and sodium valproate in patients with psychotic mania and found that personality profiles in the inpatients with psychotic mania are related to the responses to sodium valproate, but are irrelevant to the responses to lithium carbonate. Shafti (East Asian Arch Psychiatry. 2018 September; 28(3):80-84) describes that lithium carbonate was more effective than aripiprazole in improving manic symptoms in male patients with acute mania. Berk, et al. (Br J Psychiatry. 2017 June; 210(6):413-421) describes that in people with first-episode mania treated with a combination of lithium and quetiapine, continuation treatment with lithium rather than quetiapine is superior in terms of mean levels of symptoms during a 1-year evolution.

Schizoaffective disorder includes the presence of psychosis (hallucinations and delusions) and mood symptoms (manic and depressive symptoms). Small, et al. (J Clin Psychopharmacol. 2003 June; 23(3):223-8) describes the treatment of schizoaffective patients with clozapine and lithium who improved with lithium on CGI and PANSS total and negative symptom scales and cognitive measures. Potkin, et al. (Clin Ther. 2002 November; 24(11):1809-23) describes a tolerability study using coadministered lithium and quetiapine in schizoaffective disorder patients. Greil, et al. (Eur Arch Psychiatry Clin Neurosci. 1997; 247(1):42-50) describes a study of prophylactic efficacy of lithium and carbamazepine in schizoaffective disorder and it was found that the compounds are equipotent alternatives in maintenance treatment.

Lithium L-threonate can be used as a source of lithium, commonly used for the treatment of patients with bipolar disorder. For this purpose, the composition can be made by any pharmaceutical preparation, such as tablet, capsule, solution, suspension, emulsion, gelatum, ointment, lyophilized powder, pill, liposome and other pharmaceutical formulations. Therefore, the present invention provides for a pharmaceutical composition of an effective amount of lithium L-threonate and pharmaceutically acceptable excipients.

The composition/compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The characteristics of the lithium L-threonate include:
1. active structure of left-handed rotation,
2. stability in high thermal condition (temperature of decomposition>300° C.), and
3. dissolution in water (no dissolution in alcohol and ether).

Lithium (I) ion and L-threonic acid can form a stable coordination compound. Although the formation of the coordination is changeable in different pH value solution, there is no precipitate of lithium hydroxide even in basic condition (solution of lithium chloride or lithium sulphate forms precipitate just pH˜4).

L-threonic acid was selected as the ligand for lithium ion, because L-threonic acid can accelerate the absorption of the ion. In addition, there is no precipitate of lithium hydroxide even in small intestine (pH˜7), improving the absorptive ratio of lithium (I). Meanwhile, it is beneficial to bind with proteins so that lithium is taken into the body as the form of lithium (I). Moreover, lithium L-threonate achieves the treatment of patients through two pathways: the active lithium ion and the cooperation of L-threonic acid. According to these advantages, lithium L-threonate can be applied as a highly absorbable form of lithium (I) in the treatment of patients with bipolar disorder.

The invention is further described in detail by reference to the following experimental examples. These examples are provided for the purpose of illustration only, and are not intended to be limiting unless otherwise specified. Thus, the invention should in no way be construed as being limited to the following examples, but rather should be construed to encompass any and all variations which become evident as a result of the teaching provided herein.

### Example 1: The Synthesis of Lithium L-Threonate Through Ion Displacement Utilizing Calcium L-Threonate

The general reaction formula is:

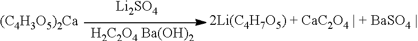

### Steps:

1. Dissolve 0.1 mol calcium L-threonate in 600 ml Water.
2. Heat the solution to 70° C. and keep 1-3 hours.
3. Add 0.1 mol oxalic crystal—producing a white precipitate of calcium oxalate.
4. Cool the mixed solution to room temperature.
5. Filter the mixed solution in decompression and collect the filtrate.
6. Concentrate the filtrate to ⅔ total volume.
7. Dissolve 33 mmol Li2SO4 in 100 ml hot Water.
8. Add the Li2SO4 solution into the concentrated filtrate of L-threonic acid.
9. Chum the mixed solution completely in 20-50° C. condition.
10. Drop 0.1 mol Ba(OH)2 into the mixed solution with a constant pressure funnel. (control the drop rate at 5mVh-15 mVh)—produce precipitate BaSO4 completely.
11. Filter rapidly the mixed solution in decompression after Ba(OH)2 dropping.
12. Concentrate the filtrate to ⅔ total volume in decompression.

Separate the concentrated filtrate to three same parts, A, B and C, continue the synthesis as follows (one of these three steps can be chosen to obtain a precipitate, the following illustrates the amounts that can be obtained with each):

### Solution A:

Drop methanol in solution A slowly with churning until the end of precipitating.
Drop another 100 ml methanol.
Filter the mixed solution.
Wash the precipitate three times with anhydrous methanol.
Wash the precipitate twice with anhydrous ether.
Desiccate the precipitate an hour in vacuum dryer.
Product ratio: 31%

### Solution B:

Adjust pH value of solution B to 3 with L-threonic acid.
Follow the steps as solution A.
Product ratio: 30%

### Solution C:

Adjust pH value of solution C to 7 with KOH.
Follow the steps as solution A.
Product ratio: 32%

### Example 2: The Synthesis of Lithium L-Threonate:Calcium L-Threonate-Lithium L-Threonate

The general reaction formula is:

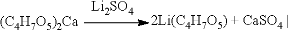

### Steps:

1. Dissolve 0.1 mol calcium L-threonate in 600 ml Water.
2. Heat the solution to 70° C. and keep 1-3 hours.
3. Dissolve 33 mmol LiSO4 in 100 ml hot Water.
4. Add the LiSO4 into the solution of calcium L-threonate.
5. Churn the mixed solution 4-7 hours.
6. Cool the mixed solution slowly to room temperature.
7. Filter the mixed solution.
8. Concentrate the filtrate to ⅔ total volume.
9. Filter again.
10. Concentrate the filtrate to 100 ml.
11. Separate the concentrated filtrate to three same parts, A, B and C, then follow the steps as Example 1.
12. The total product ratio is 88%.

### Example 3: The Synthesis of the Lithium L-Threonate:L-Threonic Acid-Lithium L-Threonate

The general reaction formula is:

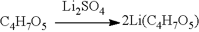

### Step I. The Synthesis of L-Threonic Acid:

The reaction formula is:

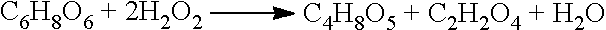

1. Dissolve 4 g calcium carbonate in 100 ml solution of 0.2 mol/L vitamin C.
2. Drop 10 ml 30% H2O2 in the mixed solution.
3. Keep the reaction in 30-40° C. condition 1 hour with churning.
4. Add 1.5 g active carbon in and heat the system to 80° C. to destroy ho.
5. Filter the solution while the system is hot to obtain a filtrate.
6. Concentrate the filtrate to ⅓ total volume in decompression at 40° C.
7. Add moderate methanol and keep statically for several hours to precipitate calcium L-threonic acid microcrystal, then filter the mixed solution.
8. Wash the microcrystal twice with 80% methanol, then transfer it to a flask.
1) Adjust pH value to 5-7 by dropping dilute oxalic acid (sulphuric acid or phosphoric acid). Filter the solution. Keep static to precipitate the crystal of L-threonic acid. The product ratio is 89%.
2) Go to ion-exchange with LR-120 (H+) ion-exchange resin. Collect, concentrate and keep the solution static to precipitate the crystal of L-threonic acid. The product ratio is 93%.

### Step II. The Synthesis of the Lithium L-Threonate:

1. Dissolve 0.1 mol L-threonic acid in 200 ml Water to form an L-threonic acid solution.
2. Dissolve 33 mmol Li2SO4 in 100 ml hot Water to form a Li2SO4 solution.
3. Add the Li2SO4 solution in the L-threonic acid solution to form a mixed solution, mix the solution completely and keep it in 30-40° C. condition.
4. Drop 0.1 mol Ba(OH)2 in the mixed solution with a constant pressure funnel, controlling the drop rate at 5 ml/h-15 ml/h, to produce precipitate BaSO4 completely.
5. Filter rapidly the mixed solution in decompression after Ba(OH)2 dropping.
6. Concentrate the filtrate to ⅓ total volume in decompression, and separate it to three same parts, A, B and C, then follow the steps as Example 1.

The product ratio is 86%.

Throughout this application, various publications, including United States patents, are referenced by author and year and patents by number. Full citations for the publications are listed herein. The disclosures of these publications and patents in their entireties are hereby incorporated by reference into this application in order to more fully describe the state of the art to which this invention pertains.

The invention has been described in an illustrative manner and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Sanacora G. New understanding of mechanisms of action of bipolar medications. J Clin Psychiatry. 2008; 69(suppl 5)-22-27.
2. Aubry J M, Schwald M, Ballmann E, Karege F. Early effects of mood stabilizers on the Akt/GSK-3beta signaling pathway and on cell survival and proliferation. Psychopharmacology (Ber). 2009; 205:419-429.
3. Einat H, Yuan P, Gould T D, et al. The role of the extracellular signal-regulated kinase signaling pathway in mood modulation. J Neurosci. 2003; 23:7311-7316.
4. Bauer L A. Applied Clinical Pharmacokinetics. 2nd Ed. New York, NY: McGraw Hill, 2008:548.
5. Kuo C C, Chen R S, Lu L, Chen R C. Carbamazepine inhibition of neuronal Na+ currents: quantitative distinction from phenytoin and possible therapeutic implications. Mol Pharmacol. 1997; 51:1077-1083.
6. Equetro [package insert]. Parsippany, NJ: Validus Pharmaceuticals, Inc.; 2007.
7. Tegretol [package insert]. East Hanover, NJ: Novartis Pharmaceuticals Corporation; 2009.
8. Depakote [package insert]. North Chicago, IL: Abbott Laboratories; 2006.
9. Goodwin G M, Bowden C L, Calabrese J R, et al. A pooled analysis of 2 placebo-controlled 18-month trials of lamotrigine and lithium maintenance in bipolar I disorder. J Clin Psychiatry. 2004; 65:432-441.
10. Ananth J. Treatment approaches to mania. Int Pharmacopsychiatry. 1976; 11:215-231.
11. Hirschfeld R M. History and evolution of the monoamine hypothesis of depression. J Clin Psychiatry. 2000; 61(suppl 6):4-6.
12. Hass E A. A Historical Look at Drug Introductions on a Five-Country Market: A Comparison of the United States and Four European Countries (1960-1981) Rockville, MD: US Food and Drug Administration Archives; March, 1982. OPE Study 60.
13. Miyake N, Otsuka Y, Kurata T. Biosci Biotechnol Biochem. Autoxidation Reaction Mechanism for I-Ascorbic Acid in Methanol without Metal Ion Catalysis. 1997 January; 61(12)-2069-75.
14. https://patents.google.com/patent/US20080248100A1
15. https://patents.google.com/patent/U.S. Pat. No. 6,077,872A
16. https://patents.google.com/patent/U.S. Pat. No. 6,313,170B1
17. Slutsky I, Abumaria N, Wu L J, Huang C, Zhang L, Li B, Zhao X, Govindarajan A, Zhao M G, Zhuo M, Tonegawa S, Liu G. Enhancement of learning and memory by elevating brain magnesium. Neuron. 2010 Jan. 28; 65(2):165-77. doi: 10.1016/j.neuron.2009.12.026.
18. Rowe D J, Ko S, Tom X M, Silverstein S J, Richards D W. Enhanced production of mineralized nodules and collagenous proteins in vitro by calcium ascorbate supplemented with vitamin C metabolites. J Periodontol. 1999 September; 70(9)-992-9.
19. Lin X M, Tian W, Ma L, Long Z, Zhang J, Shen X Y, Zhang X P. The responses of serum transferrin receptors to iron supplements in subjects with iron-deficiency erythropoiesis and iron-deficiency anaemia. Br J Nutr. 2008 February; 99(2):416-20. Epub 2007 Sep. 10.

What is claimed is:

1. A composition comprising lithium L-threonate.

2. The composition of claim 1, wherein said composition has a left-handed rotation.

3. The composition of claim 1, wherein said composition has a temperature of decomposition of greater than 300 degrees C.

4. The composition of claim 1, wherein said composition is dissolvable in water and does not dissolve in alcohol and ether.

5. A pharmaceutical composition of an effective amount of lithium L-threonate and pharmaceutically acceptable excipients.

6. The pharmaceutical composition of claim 5, wherein said composition is in a dosage form chosen from the group consisting of tablet, capsule, solution, suspension, emulsion, gelatum, ointment, lyophilized powder, pill, and liposome.

7. A method of making lithium L-threonate, including the steps of:
reacting calcium L-threonate with Li2SO4 and Ba(OH)2; and
producing lithium L-threonate.

8. The method of claim 7, wherein said reacting step is further defined as:
dissolving calcium L-threonate in water to form a solution;
heating the solution;
adding oxalic crystal and producing a precipitate of calcium oxalate;
cooling the solution;
filtering the solution and collecting a filtrate;
concentrating the filtrate;
dissolving Li2SO4 in water to form an Li2SO4 solution;
adding the Li2SO4 solution into the concentrated filtrate to form a mixed solution;
churning the mixed solution;
dropping Ba(OH)2 into the mixed solution;
filtering the mixed solution in decompression;
concentrating a filtrate; and
separating the concentrated filtrate into three equal parts of solution.

9. The method of claim 8, further including, after said separating step, a step chosen from the group consisting of:
1) dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate;
2) adjusting a pH value of the solution to 3 with L-threonic acid, dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate; and
3) adjusting a pH value of the solution to 7 with KOH, dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate.

10. A method of making lithium L-threonate, including the steps of:
reacting calcium L-threonate with Li2SO4; and
producing lithium L-threonate.

11. The method of claim 10, wherein said reacting step is further defined as:
dissolving calcium L-threonate in water to form a solution;
heating the solution;
adding LiSO4 to the solution;
churning the solution;
cooling the solution;
filtering the solution and collecting a filtrate;
concentrating the filtrate;
filtering the filtrate and collecting a second filtrate;
concentrating the second filtrate; and
separating the concentrated filtrate into three equal parts of solution.

12. The method of claim 11, further including, after said separating step, a step chosen from the group consisting of:
1) dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate;
2) adjusting a pH value of the solution to 3 with L-threonic acid, dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate; and
3) adjusting a pH value of the solution to 7 with KOH, dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate.

13. A method making lithium L-threonate, including the steps of:
synthesizing L-threonic acid;
reacting the L-threonic acid with Li2SO4; and
producing lithium L-threonate.

14. The method of claim 13, wherein said synthesizing step is further defined as:
dissolving calcium carbonate in a solution of vitamin C;
dropping H2O2 in the solution;
churning the solution;
adding active carbon to the solution and heating the solution;
filtering the solution and obtaining a filtrate;
concentrating the filtrate;
adding methanol and precipitating calcium L-threonic acid microcrystal;
filtering the solution;
washing the microcrystal and transferring to a flask; and
performing a step chosen from the group consisting of 1) adjusting pH of the solution to 5-7, filtering the solution, and precipitating L-threonic acid crystal, and 2) performing an ion-exchange with the solution, collecting the solution, and precipitating L-threonic acid crystal.

15. The method of claim 14, wherein said reacting step is further defined as:
dissolving L-threonic acid in water to form a L-threonic acid solution;
dissolving Li2SO4 in water to form a Li2SO4 solution;
adding the Li2SO4 solution to the L-threonic acid solution and forming a mixed solution;
dropping Ba(OH)2 into the mixed solution;
filtering the mixed solution in decompression;
concentrating a filtrate; and
separating the concentrated filtrate into three equal parts of solution.

16. The method of claim 15, further including, after said separating step, a step chosen from the group consisting of:
1) dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate;
2) adjusting a pH value of the solution to 3 with L-threonic acid, dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate; and
3) adjusting a pH value of the solution to 7 with KOH, dropping methanol with churning until precipitating ends, dropping additional methanol to form a mixed solution, filtering the mixed solution to form a precipitate, washing the precipitate three times with anhydrous methanol, washing the precipitate twice with anhydrous ether, and desiccating the precipitate.

17. A method of treating bipolar disorder, including the steps of:
administering an effective amount of lithium L-threonate to a patient; and
treating bipolar disorder.

18. The method of claim 17, wherein bipolar disorder is chosen from the group consisting of Bipolar Disease, Bipolar I Disorder, Bipolar II Disorder, Bipolar Depression, and Bipolar Mania.

19. The method of claim 17, further including the step of administering a compound chosen from the group consisting of mood stabilizers, atypical antipsychotics, antidepressants, and anxiolytics.

20. The method of claim 17, wherein said administering step is further defined as administering 300-2000 mg of lithium L-threonate per day.

21. The method of claim 17, wherein L-threonic acid provides accelerated absorption of lithium ion.

22. A method of treating a disease or disorder, including the steps of:
administering an effective amount of lithium L-threonate to a patient; and
treating the disease, wherein the disease is chosen from Lewy-body dementia (LBD), Alzheimer's disease (AD), Alzheimer's disease related dementias (AD/ADRD), behavioral and psychological symptoms of dementia (BPSD), agitation in Alzheimer's disease (AAD), mania, or schizoaffective disorder.

23. The method of claim 22, wherein said administering step is further defined as administering 300-2000 mg of lithium L-threonate per day.

24. The method of claim 22, wherein L-threonic acid provides accelerated absorption of lithium ion.
